(19) United States
(12) Patent Application Publication
(10) Pub. No.: US 2004/0260351 A1
(43) Pub. Date: Dec. 23, 2004

(54) Evoked response detector

(76) Inventors: Nils Holmstrom, Jarfalla (SE); Anders Bjorlinging, Jarfalla (SE)

Correspondence Address: SCHIFF HARDIN LLP, Patent Department, 6600 Sears Tower, 233 South Wacker Drive, Chicago, IL, 60606, US

(21) Appl. No.: 10/875,130

(22) Filed: Jun. 23, 2004

(30) Foreign Application Priority Data
Jun. 23, 2003 (SE) 0301800-9

(51) Int. Cl. A61N 1/37

(52) U.S. Cl. 607/27

(57) ABSTRACT

An evoked response detector for a biventricular pacing system has a measuring arrangement for measuring a cardiac parameter signal indicative of mechanical contraction of the heart after a biventricular stimulation. A comparator compares the parameter signal, in a time window of predetermined length after the stimulation, with a number of predetermined stored parameter signal templates representing different capture situations to determine most similar template and thus the existing capture situation.

### BACKGROUND OF THE INVENTION

1. Field of the Invention

The present invention relates to an evoked response detector for a biventricular pacing system.

1. Description of the Prior Art

U.S. Pat. No. 6,148,234 discloses a dual site pacing system, either biventricular or biatrial, wherein the pacemaker looks for a signal sensed during the refractory period following delivery of the pulse pair. If the threshold of either head chamber has risen above the level of the delivered pulses, that chamber will not be captured and will not have an inherent refractory period following the delivery of the pulse pair. For patients having conduction delay from one chamber to the other, e.g. LBBB or RBBb the signal from the other chamber will then be sensed in the non-captured chamber during the pacemaker refractory period. Such a sensing is recognized to result from loss of capture and is utilized to automatically increase the pulse amplitude to a safe level above threshold.

U.S. Pat. No. 5,713,933 describes circuitry provided in an ordinary pacemaker for monitoring the cardiac impedance waveform during a predetermined capture detect window following delivery of stimulating impulses. One or more values are derived characterizing the morphology of the impedance waveform during the capture detect window associated with each stimulation pulse delivered. These values are compared to predetermined control values in order to assess whether the stimulation pulse has resulted in cardiac capture in order to determine the patients'cardiac stimulation threshold.

U.S. Pat. No. 6,512,953 describes detection of capture during multi-chamber stimulation from intracardiac electrogram (IEGM) signals measured from selected pairs of chambers subsequent to delivery of stimulation pulses. The measured IEGM signals are compared with stored IEGM characteristics representing non-capture in both chambers, single-chamber capture and bi-chamber capture to distinguish between these three situations of capture.

For biventricular pacing it is difficult to detect capture at the first stimulated ventricle, because the stimulation pulse in the second ventricle normally will occur inside the evoked response detection window ERDW of the first ventricle. This problem is illustrated in FIG. 1, where A1 represents a first atrium, V1 the first stimulated ventricle, and V2 the second ventricle. If the first stimulated atrium A1 is, e.g., the right atrium, the first stimulated ventricle V1 need not necessarily be the right ventricle. A P-wave P1 is shown on Al and after the P-V time delay a stimulation pulse is delivered to the first ventricle, V1, resulting in a response 4 within the corresponding ERDW. A stimulation pulse 6 is delivered to the other ventricle V2 after a time-delay of V1-V2 after the stimulation pulse 2 to the first ventricle V1. The corresponding response 8 in the second ventricle V2 appears in ERDW of V2. As appears from FIG. 1, the stimulation pulse to the second ventricle V2 is emitted within the evoked response detection window ERDW of the first ventricle V1.

### SUMMARY OF THE INVENTION

An object of the present invention is to provide an evoked response detector solving the above mentioned evoked response detection problem at the ventricular pacing.

The above object is achieved in accordance with the invention by an evoked response detector producing a biventricular pacing system, wherein the evoked response detector has a measuring arrangement for measuring a cardiac parameter signal indicative of mechanical contraction of the heart after a biventricular stimulation, and a comparator that compares the parameter signal, in a time window of predetermined length after the stimulation, with a number of predetermined stored parameter signal templates respectively representing different capture situations. The comparator determines the most similar template, and thus the existing capture situation.

The above object also is achieved in accordance with the invention by a biventricular pacing system having an evoked response detector as described above.

With the detector according to the present invention, it is possible to distinguish between capture, loss of capture and some degree of fusion. It is also possible to distinguish left ventricular capture and right ventricular loss of capture from the opposite situation. As a parameter indicative of heart contraction the electrical impedance preferably is used, but several mechanical indicators of heart contraction can be used for this purpose as well. For the impedance measurements standard pacing leads can be used to detect the mechanical contraction of the heart. Unipolar impedance measurements or bipolar impedance measurements preferably are used in both ventricles. It is, however, also possible to perform impedance measurements between the right and left ventricles.

In an embodiment of the detector according to the invention, the measuring arrangement is a pressure measuring arrangement for measuring a pressure signal from at least one of the ventricles after a biventricuiar stimulation and a comparator compares the pressure signal with a number of predetermined stored pressure signal templates representing different capture situations to determine the most similar template and thus the existing capture situation. The measuring arrangement alternatively can be a pressure measuring arrangement for measuring a pressure signal from the arteries for the same purpose. Depending on the position in the heart, the pressure signal morphology can be used as an indicator of capture in right, left or both ventricles. When the heart's ventricles are stimulated, contractions will follow after a few milliseconds. During the isovolumetric contraction phase the pressure increases rapidly after a specific time in both ventricles. If only one ventricle is captured there will be a time lag between pressure increases and the sequence of the slopes indicates in which ventricle the loss occurred.

The time derivative of the pressure, dp/dt, can be used as a parameter indicative of heart contraction as well.

### DESCRIPTION OF THE DRAWINGS

FIG. 1, as described above, is a time diagram illustrating the problem associated with evoked response detection in the context of biventricular stimulation.

FIG. 2 illustrates an impedance measurement arrangement for the right and left ventricles, suitable for use in the evoked response detector according to the invention.

FIG. 3 is a time diagram illustrating capture on both ventricles.

FIG. 4 is a time diagram illustrating loss of capture on the first ventricle and capture on the second ventricle.

FIG. 5 illustrates a capture situation on the first ventricle and loss of capture on the second ventricle.

FIG. 6 illustrates the capture situation of no capture on any ventricle.

FIG. 7 illustrates the comparison of measured impedance signals with templates in accordance with the invention, in an embodiment using the least squares method.

FIG. 8 illustrates an alternative technique for comparing the measured impedance signals with templates, in accordance with the invention.

FIG. 9 schematically illustrates the lead of a conventional biventricular pacing system.

FIG. 10 is an embodiment of an impedance measurement arrangement and a comparator in the evoked response detector according to the invention.

FIG. 11 shows impedance signals and pressure signals respectively as functions of time after stimulation of the right and left ventricles.

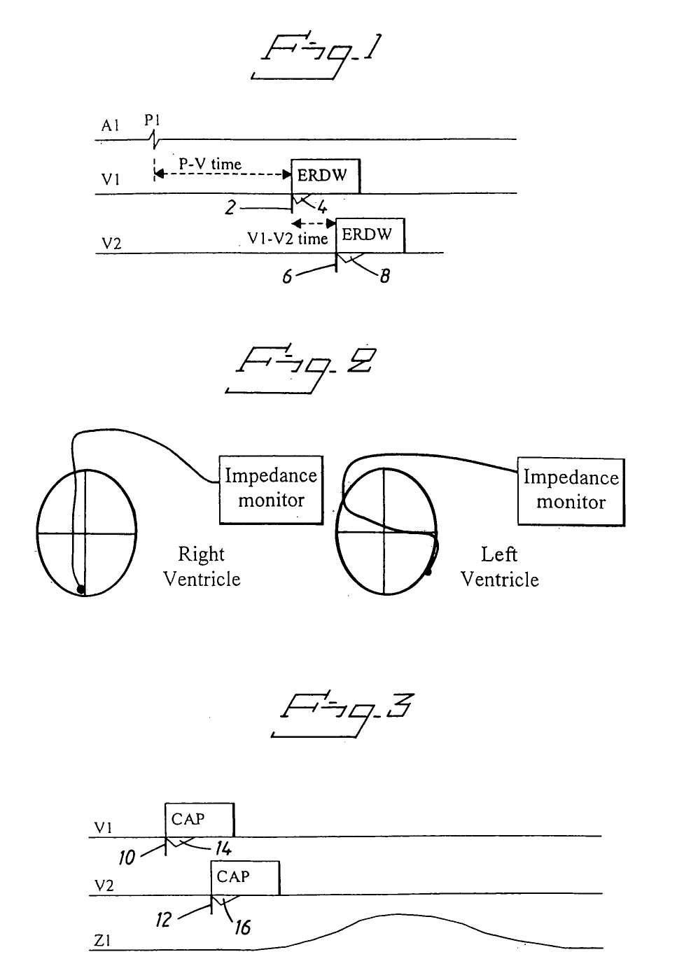

### DESCRIPTION OF THE PREFERRED EMBODIMENTS

In an embodiment of the detector according to the invention, the impedance morphology IM is utilized for determining if and which one of stimulation pulses captured each ventricle, respectively. FIG. 2 shows an example of the impedance measurements. Unipolar, i.e. tip-case or ring-case, impedance configurations and bipolar, i.e. tip-ring, impedance configurations, can preferably be used in both ventricles for measuring the impedances Z1 and Z2, but also impedance measurements between the right and left ventricle can be used. The measurements can typically last for 330 ms after an emitted pacing pulse. By comparing the impedance signals with pre-recorded patient individual templates the result of the pacing pulses can be determined.

Patient-dependent individual impedance templates are created as follows.

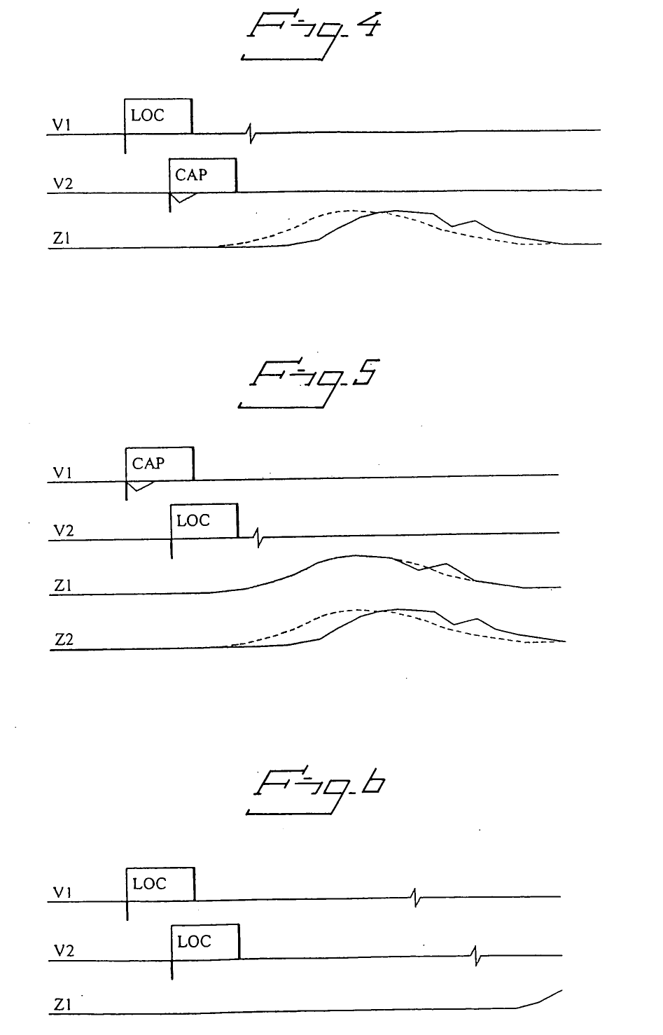

The amplitudes of the two pacing pulses are set to such a high value that capture is guaranteed, preferably equal to a maximum amplitude value of typically 4.5 V. In this way capture on both ventricles is secured. The impedance is measured for e.g. 5 consecutive pacing cycles and a mean value is stored as the double capture template. This situation is illustrated in the diagram on FIG. 3. A pacing pulse 10 to the first ventricle V1 results in capture and a pacing pulse 12 to the second ventricle V2 results in capture 16 as well. With capture on both ventricles with optimal hemodynamics the measured impedance Z1 has a typical morphology as illustrated in FIG. 3.

To create a loss-capture template the pacing pulse to the first ventricle V1 is omitted, whereas the amplitude to the second ventricle is maintained at a sufficiently high value of typically 4.5 V to secure capture. This situation is illustrated in FIG. 4. After loss of capture LOC on the first ventricle V1 the contraction starts later and the IM pattern alters accordingly to asynchronous contraction as illustrated by the solid curve Z1 in FIG. 4 which represents the mean (average) of the impedance signal which then is stored as the loss-capture template. The dashed Z1 curve in FIG. 4 represents the capture-capture template according to FIG. 3.

To create the capture-loss template the stimulation pulse to the second 20 ventricle V2 is omitted while the pulse to the first ventricle V1 is kept equal to 4.5 V. The mean value of the impedance signals during e.g. 5 pacing cycles is formed and stored as capture-loss template, c.f., FIG. 5. Thus, as appears from FIG. 5, if the local Mat the second ventricle V2, solid 72 line, is delayed compared to the IM at the first ventricle, solid Z1 line, this is an indication of LOC on the second ventricle, cf FIG. 5.

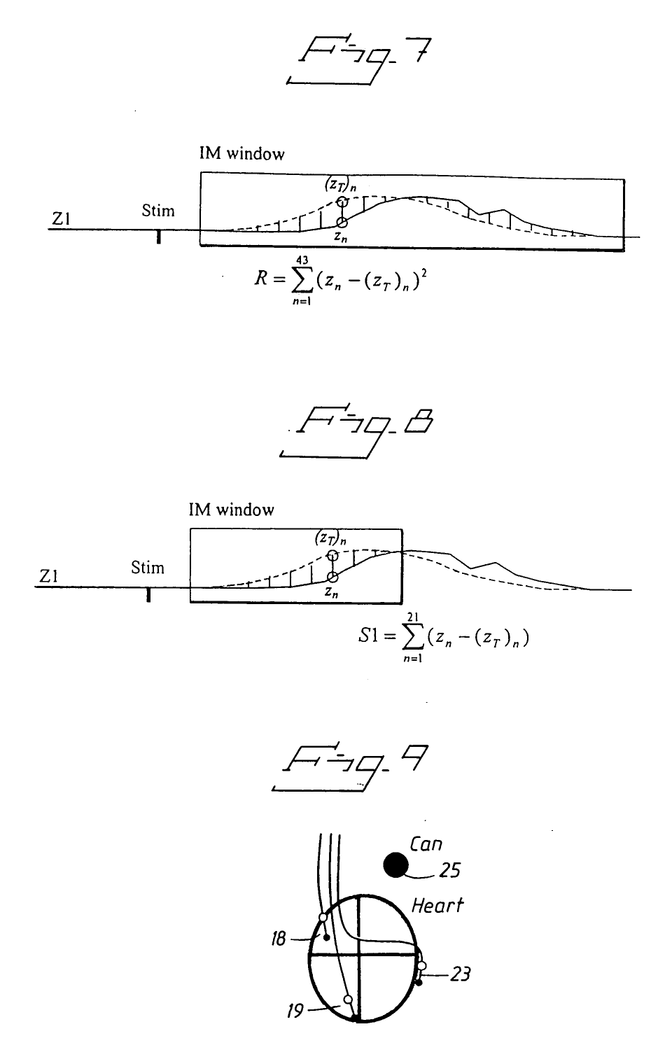

To create loss-loss template both pacing pulses are inhibited and the resulting IM is stored as loss-loss template, cf FIG. 6. As can be seen from FIG. 6 there is no contraction to be observed in the measured impedance Z1 in case of LOC on both ventricles. FIG. 6 also shows the impedance Z1 beginning at a sensed R-wave, which is stored as a template of an intrinsic contraction.

After each biventricular stimulation, i.e. the emission of two pacing pulses, the impedance is measured and the impedance signal is compared to the five predetermined templates mentioned above and the difference between template and measured impedance signal is determined using the least square method, as illustrated in FIG. 7, or other suitable method. Thus, the difference between the actual measured IM, solid line in FIG. 7, and the template, dashed line in FIG. 7, is determined according to the equation

$$R = {\sum }_{n = 1}^{43} {( {Z}_{n} - {( {Z}_{T} )}_{n} )}^{2}$$

where Zₙ denotes sample values of the impedance signal, (Z_{T})ₙ sample values of the template in question and 43 is the total number of samples for a window length of 330 ms and a sampling frequency of the impedance equal to 128 Hz. The value of R can then be used to distinguish between different capture and fusion situations by comparing the difference R with predetermined threshold values both for the first and the second ventricle.

The decisions based on the difference or distance between measured signal and the individual templates are summarized in the following table.

| Most similar template | Device decision |
|---|---|
| Capture-capture template | Capture on both ventricles |
| Capture-loss template | Capture on the first ventricle, loss of |
| | Capture on the second one |
| Loss-capture template | Loss of capture on the first ventricle, |
| | capture on the second one |
| Loss-loss template | Loss on both ventricles |

If loss is detected, on any or both of the ventricles, this might be caused by fusion. The distance to the intrinsic contraction template determines the degree of fusion. If the pacing sequence was determined to loss-loss, LL, loss-capture, LC, or capture-loss, CL, and the distance to the intrinsic contraction template is small, there is a high possibility that the beat is a fusion beat. For decreasing similarity to the intrinsic contraction template, the possibility of fusion is decreasing and the possibility of a real loss is increased.

As an alternative method for studying capture and loss of capture, the difference between measured impedance signals and templates are studied only during the first half of the impedance measurement window, the length of which typically amounting to 150 ms, and summarize this difference S1. The difference S1 is then given by the equation

$$S1 = {\sum }_{n = 1}^{21} ( {Z}_{n} - {( {Z}_{T} )}_{n} )$$

where Zₙ denotes the nth sample value of the impedance signal, (Z_{T})ₙ the nth sample value of the template in question and 21 denotes the upper limit of this first half of the IM window, obtained for a window of the length 150 ms and a sampling frequency of 128 Hz, as illustrated in FIG. 8. If S1<0 the measured impedance signal is delayed indicating loss of capture. If S1>0 the measured impedance signal increases before the template indicating e.g. increased workload.

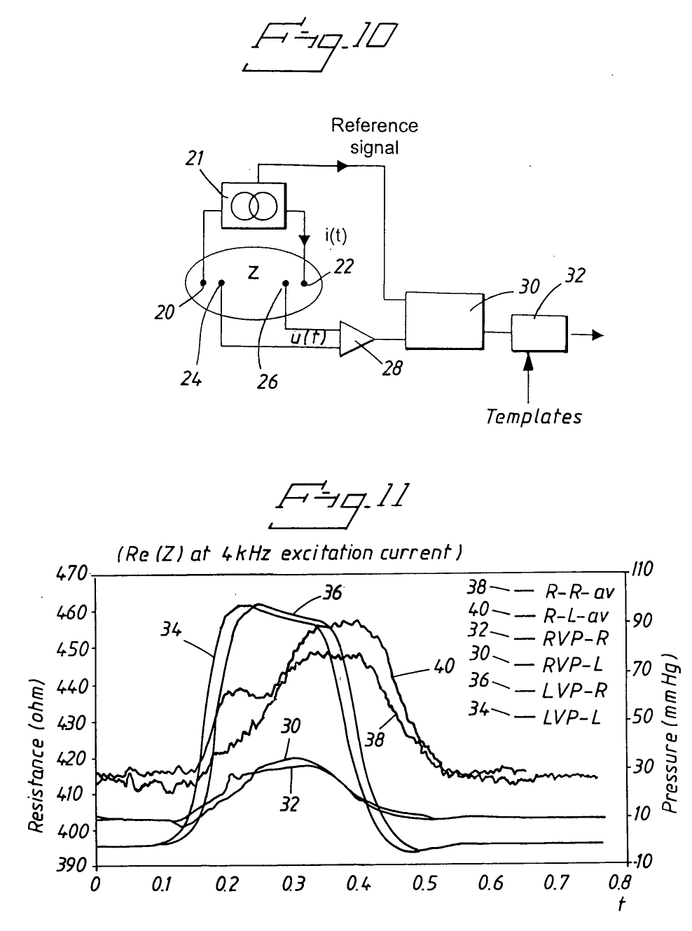

As indicated above, the impedance can be measured at several places. By use of e.g. bipolar leads 18 in the right atrium, the right ventricle 19 and in the left coronary vein 23, access is obtained to several electrodes including the pulse generator can 25, see FIG. 9. If an electric current is supplied to two of the electrodes an evoked voltage response can be measured between any combination of pairs of electrodes.

With reference to FIG. 10, an electric excitation current i(t) is supplied to two electrodes 20, 22 and the resulting voltage u(t) is measured between two measurement electrodes 24 and 26. The measured voltage signal u(t) is amplified in an amplifier 28 and the amplified voltage signal u(t) is synchronized with the current i(t) with the aid of a reference signal picked up from the current source 21 and supplied to synchronizing means 30. The resulting impedance is given by the expression

Z=u/i

This measured impedance Z is then compared to predetermined, stored templates in a comparator 32 to determine the actual capture situation.

Above, embodiments have been described using the cardiac impedance signal to monitor the mechanical contraction of the head. Other mechanical indicators of heart contraction can, however, be used for that purpose as well. Thus, the pressure profile in e.g. the chambers of the heart and even in the arteries can be used as indicator of capture. Also the acceleration of the myocardium can be used as parameter for mechanical evoked response.

A sensor with an output signal reflecting the rate of change of the pressure is useful for this purpose. Depending on position in the head, the pressure signal morphology can be used as indicator of capture in right, left or both ventricles. When the hearts ventricles are stimulated, contractions will follow after a few milliseconds. During the isovolumetric contraction phase, the pressure increases rapidly after a specific time in both ventricles. If only one ventricle is captured, there will be a time lag between the pressure raises and the order of the slopes tells in which ventricle the loss of capture occurred.

FIG. 11 shows an example of pressure and impedance signals when the right and left ventricles are stimulated, at time t=0 in the FIG. 11, a 4 kHz excitation current being used for the impedance measurements. Curve 38 shows the impedance signal when the right ventricle is stimulated and curve 40 the impedance signal when the left ventricle is stimulated. Curve 32 shows the right ventricular pressure when the right ventricle is stimulated and curve 30 the right ventricle pressure when the left ventricle is stimulated. Curve 36 shows left ventricular pressure when the right ventricle is stimulated and curve 34 the left ventricular pressure when the left ventricle is stimulated.

The technique described above can as well be applied to single ventricular dual chamber or single chamber systems. The number of possible templates will then be less.

Although modifications and changes may be suggested by those skilled in the art, it is the intention of the inventor to embody within the patent warranted hereon all changes and modifications as reasonably and properly come within the scope of his contribution to the art.

1. An evoked response detector for a biventricular pacing system, comprising:
a measuring arrangement adapted for interacting with a heart for measuring a cardiac parameter signal indicative of mechanical contraction of the heart after a biventricular stimulation; and
a comparator arrangement for comparing said parameter signal, in a time window of predetermined length after said biventricular stimulation, with a plurality of predetermined stored parameter signal templates respectively representing different capture situations, for determining a most similar template along said plurality of templates, and thereby also determining an existing capture situation.

2. An evoked response detector as claimed in claim 1, wherein said measuring arrangement comprises an impedance measuring arrangement for measuring an impedance signal, at least one ventricle of the heart after said biventricular stimulation, and wherein said comparator arrangement compares said impedance signal with a plurality of predetermined stored impedance signal templates respectively representing different capture situations, for determining said most similar template and said existing capture situation.

3. An evoked response detector as claimed in claim 2 wherein said impedance measuring arrangement comprises an electrical current source and supplied electrodes connected to said electric current source and adapted to interact with the heart to inject alternating electric current into each ventricle, and measuring electrodes respectively adapted for interacting with the ventricles, and a measuring unit connected to the measuring electrodes or measuring a voltage between said measuring electrodes.

4. An evoked response detector as claimed in claim 3 comprising a single set of electrodes forming both said supply electrodes and said measuring electrodes.

5. An evoked response detector as claimed in claim 3 wherein said supply electrodes and said measuring electrodes are configured as unipolar electrodes.

6. An evoked response detector as claimed in claim 3 wherein said supply electrodes and said measuring electrodes are configured as bipolar electrodes.

7. An evoked response detector as claimed in claim 3 wherein said supply electrodes are respectively adapted for implantation in the right ventricle and a coronary vein on the left side of the heart and wherein said measuring electrodes are respectively adapted for implantation in the right ventricle and a coronary vein on the left side of the heart.

8. An evoked response detector as claimed in claim 1 wherein said comparator arrangement employs a time window, as said time window of predetermined length after said biventricular stimulation, having a duration of approximately 330 msec.

9. An evoked response detector as claimed in claim 1 wherein said measuring arrangement comprises a pressure measuring arrangement adapted to interact with at least one ventricle for measuring a pressure signal from said at least one ventricle after said biventricular stimulation, and wherein said comparator arrangement compares said pressure signal with a plurality of predetermined stored ventricular pressure signal templates respectively representing different capture situations, for determining said most similar template and said existing capture situation.

10. An evoked response detector as claimed in claim 1 wherein said measuring arrangement comprises a pressure measuring arrangement adapted to interact with an artery for measuring a pressure signal from said artery, and wherein said comparator arrangement compares said pressure signal with a plurality of predetermined stored arterial pressure signal templates respectively representing different capture situations, for determining said most similar template and said existing capture situation.

11. An evoked response detector as claimed in claim 1 wherein said measuring arrangement is adapted to interact with the myocardium for measuring acceleration of the myocardium after said biventricular stimulation, and wherein said comparator arrangement compares said measured acceleration of the myocardium with a plurality of predetermined acceleration signal templates respectively representing different capture situations, for determining said most similar template and said existing capture situation.

12. An evoked response detector as claimed in claim 1 wherein said measuring arrangement measures said parameter signal in a predetermined number of cardiac cycles after delivery of stimulation pulses to both ventricles of an amplitude for obtaining capture, and wherein said evoked response detector comprises an average value former for forming an average value of the measured parameter signals in said number of cardiac cycles, and/or for storing said average value as a double-capture template.

13. An evoked response detector as claimed in claim 1 wherein said measuring arrangement said parameter signal in a predetermined number of cardiac cycles after delivery of a stimulation pulse only to a first ventricle of an amplitude for obtaining capture on said first ventricle, and wherein said evoked response detector comprises an average value former for forming an average value of said measured parameter signals in said number of cardiac cycles, and for storing said average value as a capture-loss template.

14. An evoked response detector as claimed in claim 1 wherein said measuring arrangement said parameter signal in a predetermined number of cardiac cycles after delivery of a stimulation pulse only to a second ventricle of an amplitude for obtaining capture on said second ventricle, and wherein said evoked response detector comprises an average value former for forming an average value of said measured parameter signals in said number of cardiac cycles, and for storing said average value as a loss-capture template.

15. A detector as claimed in claim 1 wherein said measuring arrangement measures said parameter signal in a predetermined number of cardiac cycles without any stimulation pulses being delivered through the heart, and wherein said evoked response detector comprises an average value former for forming an average value of the measured parameter signals in said number of cardiac cycles, and for storing said average value as a loss-loss template.

16. An evoked response detector as claimed in 1 wherein said measuring arrangement measures said parameter signal in a predetermined number of cardiac cycles after sensing an R-wave from the heart, and wherein said evoked response detector comprises an average value former for forming an average value of said measured parameter signals in said number of cardiac cycles, and for storing said average value as an intrinsic contraction template.

17. An evoked response detector as claimed in claim 1 wherein said measuring arrangement is an impedance measuring arrangement for measuring an impedance signal from at least one of the ventricles after said biventricular stimulation, and wherein said comparator arrangement calculates a difference R between said measured impedance signal and the respective templates in said plurality of templates using a least squares method according to
$$R = {\sum }_{n = 1}^{N} {( {Z}_{n} - {( {Z}_{T} )}_{n} )}^{2}$$
wherein Zₙ denotes sample values of the impedance signal, (Z_{T})ₙ denotes sample values of the template in question, and N denotes a total number of samples in said time window.

18. An evoked response detector as claimed in claim 17 wherein said comparator arrangement determines a measured impedance signal as corresponding to one of said plurality of templates if said difference R is less than a predetermined limit value.

19. An evoked response detector as claimed in claim 1 wherein said measuring arrangement is an impedance measuring arrangement for measuring an impedance signal from at least one of the ventricles after said biventricular stimulation, and wherein said comparator arrangement calculates a difference S between the measured impedance signal and the respective templates in said plurality of complex during a first part of said time window, according to
$$S = {\sum }_{n = 1}^{m} ( {Z}_{n} - {( {Z}_{T} )}_{n} )$$
where Zₙ denotes an nth sample value of the impedance signal, (Z_{T})ₙ denotes an nth sample value of the template in question, and wherein m denotes an upper limit of said first part of said time window, and wherein said comparator determines a condition S<0 as an indication of loss of capture.

20. A biventricular pacing system comprising:
a pacing circuit having stimulation electrodes adapted for interaction with both ventricles of a heart for biventricular pacing of the heart; and
an evoked response detector comprising a measuring arrangement adapted for interacting with a heart for measuring a cardiac parameter signal indicative of mechanical contraction of the heart after a biventricular stimulation, a comparator arrangement for comparing said parameter signal, in a time window of predetermined length after said biventricular stimulation, with a plurality of predetermined stored parameter signal templates respectively representing different capture situations, for determining a most similar template along said plurality of templates, and thereby also determining an existing capture situation.

21. A biventricular pacing system as claimed in claim 20 wherein said measuring arrangement has measuring electrodes, and that comprising a single set of leads adapted for the implantation respectively in the ventricles or coronary veins said leads carrying both said stimulation electrodes and said measuring electrodes.
